(19) United States
(12) Patent Application Publication
(10) Pub. No.: US 2010/0150887 A1
(43) Pub. Date: Jun. 17, 2010

(54) Tissue Progenitor Cells That Overexpress ERG

(76) Inventors: Henry J. Duff, Calgary (CA); Xiang Zhao, Calgary (CA); James Charles Cross, Calgary (CA); Guoqi Teng, Calgary (CA); James Paul Lees-Miller, Calgary (CA)

Correspondence Address: FULBRIGHT & JAWORSKI L.L.P., 600 CONGRESS AVE., SUITE 2400, AUSTIN, TX, 78701, US

(21) Appl. No.: 12/524,518

(22) PCT Filed: Jan. 23, 2008
(86) PCT No.: PCT/IB08/00957

§ 371 Date: Jan. 6, 2010

Related U.S. Application Data
(60) Provisional application No. 60/886,556, filed on Jan. 25, 2007.

(51) Int. Cl. A61K 45/00; C12N 5/0735; C12N 5/077; C12N 5/071; A61P 9/10

(52) U.S. Cl. 424/93.21; 435/325; 435/366; 435/372; 424/93.7

(57) ABSTRACT

Methods for generating a population of mesodermal progenitor cells. In some aspects pluripotent cells such as stem cells are transformed with wild-type ERG expression cassette. Mesodermal progenitor cells of the invention may be used for the treatment of cardiac damage such as myocardial infarction.

The present application claims benefit of priority to U.S. Provisional Application Ser. No. 60/886,556, filed Jan. 25, 2007, the entire contents of which are hereby incorporated by reference.

### BACKGROUND OF THE INVENTION

1. Field of the Invention

The invention generally concerns methods for differentiating pluripotent cells. More specifically, the invention concerns methods for producing populations of cells that can integrate into damaged cardiac tissue.

2. Description of Related Art

While the ERG1 gene (ERG) may be best known for its role in the heart, its function is not limited to heart. The ERG channel is overexpressed in many tumors and pharmacologic blockade of ERG decreases tumor growth (Pillozzi et al., 2002; Crociani et al., 2003; Cherubini et al., 2002; Lastraioli et al., 2004). Arcangeli et al. (1995) reported that expression of ERG is significantly up-regulated at the S phase of cell cycle and it has also been reported that HERG overexpression promotes tumor cell proliferation via a tumor necrosis factor-alpha (TNFα) signal transduction pathway (Wang et al., 2004; Wang et al., 2002). Additionally, metastasis (migration) of colonic tumor cells is directly related to ERG conductance (Lastraioli et al., Cancer Res. 64: 606-611, 2004). ERG increases adhesion-dependent neuritogenesis (outgrowth/mobility/migration) of neuroblastoma cells (Arcangeli et al., 1996; Bianchi et al., 1995; Arcangeli et al., 2004; Cherubini et al., 2005). This increased mobility likely relates to physical and functional interaction of HERG with adhesion receptors of the integrin family (Arcangeli, 2005; Hofmann et al., 2001; Cherubini et al., 2002). Cell contact and adhesion result in activation of BERG currents. HERG conductance is associated with recruitment of focal adhesion kinase (FAK) and FAK becomes tyrosine phosphorylated. Pharmacologic inhibitors of HERG block tyrosine phosphorylation of FAK (Cherubini et al., 2002). Activation of HERG conductance similarly activates the small g protein-GTPase Racl, Rho (Cherubini et al., 2005). Previous studies also confirm that cell extension of the plasmalemma during cell motility is dependent on Rho, RAC and Cdc42 (Miyashita et al., 2004). Furthermore, Puceat has reported that GTP Rac has a role in cardiac differentiation from stem cells (Puceat et al., 2003). These data indicate that ERG increases mobility, possibly through the small G proteins. ERG also provides a survival advantage in tumors by fostering development of neovascularization. Arcangeli reported that BERG currents promote production and secretion of VEGF. These observations suggest that ERG may play a variety of roles in cardiac tissue development (Masi et al., 2005).

Cardiovascular disease remains the leading cause of death in most westernized countries. Thus, new methods for treating cardiovascular disease in need. Cardiac damage in particular has proven to be a difficult treatment target since this specialized organ has a limited ability to repair regions of tissue that become damaged due to infection, drug toxicity or hypoxia (as occurs during a heart attack). One approach that is being explored in the delivery of pluripotent cardiac progenitor cells (Wollert et al., 2004; Schachinger et al., 2006). Thus far however, these methods have proven only marginally effective a repairing damaged tissue. Thus, there is currently a need for cardiac progenitor cells with an enhanced ability to repair tissue and methods for making and using such cells.

### SUMMARY OF THE INVENTION

Thus, in accordance with the present invention, there is provided a method for making mesodermal progenitor cells. Such a method may comprise (a) obtaining a progenitor cell population from a mammal, (b) transforming at least one progenitor cell with an expression cassette comprising a wt-ERG coding region under the control of a heterologous promoter, and (c) allowing the wt-ERG expressing cells to grow under permissive conditions and to differentiate into mesodermal progenitor cells. As used herein the term “mesodermal progenitor cells” refers to a population of cells that are capable of differentiating into a various mesoderm lineages such as vascular cells and cardiac cells. For example, a mesodermal progenitor cell population may be capable of differentiating into endothelial cells and/or cardiomyocytes. Thus, in certain aspects, mesodermal progenitor cells may be defined as comprising cardiomyocyte precursor cells and/or endothelial precursor cells. In still a further aspects of the invention, there is a provided a mesodermal progenitor cell or a mesodermal progenitor cell population obtained by a method of the invention. In some aspects, these cells may be further defined as human cells.

In still further aspects, methods of the invention involve obtaining a population of pluripotent mammalian cells. Pluripotent cells may be from a variety of sources for example, the cells may be embryonic stem cells. Method for obtaining murine or human embryonic stem cells are known in the art (Tang et al., 2004; U.S. Pat. No. 6,200,806). In some further aspects, a pluripotent cell population may be cord blood cells (from umbilical cord), mesenchymal cord stem cells (see, for example, Romanov et al., 2003) or bone marrow cells. Furthermore, in some aspects, cardiac stem cells may be used in methods of the invention. Cardiac stem cells, may for example, be isolated from cardiac biopsy samples of adult heart tissue, and may be classified as mesodermal progenitor cells from adult heart.

In certain aspects the invention concerns an expression cassette comprising a wild-type (wt) ERG coding region under the control of a heterologous promoter. A wt-ERG for use in the current invention may, in some aspects, be a mammalian wt-ERG such as the human ERG (HERG) sequences. In some aspects, wt-ERG may be further defined as an ERG molecule that does not comprise the N629D point mutation. Thus, in some aspects, a wt-ERG may be the HERG sequence given in SEQ ID NO:1 (NCBI accession no. ABF71886). In further aspects, the expression of wt-ERG is under the control of a heterologous promoter sequence. Such as sequence may be any promoter other than an ERG promoter, for example one of the promoter selected from Table 1. For instance, in some cases, the promoter is a viral promoter or an inducible promoter (e.g., one of the inducible promoter of Table 2). Thus, a wt-ERG expression cassette of the invention may comprise a CMV promoter or a tetracycline inducible or repressible promoter. Furthermore, an expression cassette of the invention may comprise additional sequence that facilitate expression of HERG including by not limited to splice sites, a polyadenylation signal, a promoter enhancer element or an insulator sequences. Thus, in some aspects, a wt-ERG expression cassette may be provided as plasmid as plasmid or as a viral vector. A nucleic acid comprising the expression cassette may comprise additional sequences such as a reporter gene or a drug resistance marker and additional sequence that for the expression of such genes. For example, a nucleic acid may comprise a green fluorescence protein gene or a gene conferring resistance to a drug such as G418. Furthermore, a nucleic acid comprising a wt-ERG expression cassette may comprise sequences that mediate integration into a host cell genome. For example, sequences that are homologous to one or more regions of a host cell genome and therefore promote homologous recombination in the host cell. Thus, in a very specific aspect of the invention, a wt-ERG expression cassette may comprise wt-hHRG gene and a GFP gene under the control of a CMV-IE chicken β-actin promoter.

Methods for transforming mammalian cells, such as pluripotent cells, are known in the art. For example, cells may be transfected using chemicals such as calcium phosphate or liposome delivery vehicles may be employed. Furthermore, in some instances, cells may be electroporated. In yet further aspects, the cell may be transduced using a viral vector. Such a method will depend upon the ability of the viral vector to efficiently enter the target pluripotent cells and thereby express the gene of interest (wt-ERG). Nonetheless, it is contemplated that vectors such as adenovirus or retrovirus vectors may be used for such a purpose. For example, a lentiviral vector may be used to transform a pluripotent cell. Method for transducing pluripotent cells and vectors for used in such transduction methods have been described, for instance in U.S. Patent Publ. 2003000874, incorporated herein by reference.

In a further embodiment, the invention provides a method for making mesodermal progenitor cells comprising the steps of (a) obtaining a pluripotent cell population from a mammal, (b) transforming/transfecting at least one progenitor cell with an expression cassette comprising a wt-ERG coding region under the control of a heterologous promoter, (c) selecting transformed cells that comprise the expression cassette (e.g., to generate s clonal cell line), and (d) allowing the cells grow under permissive conditions and thereby differentiate into mesodermal progenitor cells (e.g., cardiac and vascular progenitor cells). Thus, in some cases, the step of selecting a transformed cell(s) may comprise selecting cells wherein the wt-ERG expression cassette is stably expressed in the cell or integrated into the genome of the cell. Furthermore, in certain aspects a cell transformed with a wt-ERG expression cassette may be selected by exposing the cells to a cytotoxic drug. In these aspects, a wt-ERG expression cassette may additional comprise sequences that encode (and facilitate expression of) a drug resistance marker. Alternatively, a drug resistance marker expression vector may be cotransformed into a cell along with a wt-ERG expression cassette. Thus, in some cases a drug selection may be used to select cells comprising a genome integrated wt-ERG expression cassette. In still further aspects, cells comprising a wt-ERG expression cassette may be selected based upon expression of a report gene. For instance, a reporter gene expression cassette may be encoded on the same nucleic acid molecule as the wt-ERG expression cassette or cotransformed into a cell with the wt-ERG expression cassette. In certain aspects, a reporter gene may be fluorescence gene such as GFP, RFP or YFP. Thus, in some aspects, selecting a cell comprising a wt-ERG expression cassette may be further defined as selecting a cell comprising a fluorescent reporter gene. Such methods may be accomplished, for example, by fluorescence assisted cell sorting (FACS).

In some aspects, methods of the invention concern allowing the ERG expressing cells to grow under permissive conditions in order to differentiate into mesodermal progenitor cells. Such methods of cell culture are well known in the art. For examples, in some aspects the cells may be grown in a medium lacking leukemia inhibitory factor. In some aspects, such a medium may comprise additional cytokines or growth factors to further stimulate proliferation or differentiation of the cells. Furthermore, in some cases, permissive culture conditions may comprise physical agitation of the cells such as rocking or stirring of the cells/medium. Furthermore, in some aspects the cells may be co-cultured with stromal cells (e.g., OP9 cells) or cultured in a stromal cell conditioned media to provide supportive paracrine and endocrine signaling. Furthermore, some aspects of the invention involve the growth of cells on solid or gel supports, such as in a gelatin matrix.

In some further embodiments, the invention provides a mammalian cell comprising an expression cassette, the expression cassette comprising a wt-ERG coding region under the control of a heterologous promoter integrated into the genome of the cell. In some aspects, the mammalian cell may be further defined as pluripotent precursor cell or a stem cell (e.g., an embryonic, adult stem cell or adult progenitor cell). In some cases, such pluripotent cell expressing wt-ERG defined as a mesodermal progenitor cell. Thus, in some aspects, there is provided a mesodermal progenitor cell comprising an expression cassette, the expression cassette comprising a wild-type (wt) ERG coding region under the control of a heterologous promoter integrated into the genome of the cell.

In some aspects, a mesodermal progenitor cell of the invention may be defined as a cell that expresses increased levels of wt-ERG polypeptide relative to a normal cardiomyocyte or relative to an untransformed stem cell. For example, a mesodermal progenitor cell may express about two, three, four, five or more times as much wt-ERG as a normal cardiomyocyte or pluripotent cell. Methods for determining the amount wt-ERG expressed in a cell are well known in the art, for example such measurements may be made using wt-ERG-binding antibodies to perform a Western blot. In certain other aspects, the amount of wt-ERG in a cell may be assessed by ELISA. Thus, in some cases, mesodermal progenitor cell of the invention may comprise more wt-ERG polypeptide per cell or per microgram of total protein than a normal cardiomyocyte or a pluripotent cell. As used herein the term normal cell means a non-transgenic cardiomyocyte.

A mesodermal progenitor cell of the invention may be further defined as comprising marker for a particular lineage. For example, in some aspects, a mesodermal progenitor cell comprises a myocardial cell marker such as myosin heavy chain expression. In still further aspects, a mesodermal progenitor cell may comprise endothelial cell markers such CD31 expression. In yet further aspects, there is provided a population of mesodermal progenitor cells. Such a population of progenitor cells may in some cases comprise cells with endothelial lineage markers and cells with myocardiocyte markers. Furthermore, in certain aspects, a mesodermal progenitor cell population may be defined by its functional characteristics. For example, the population may comprise beating embryoid bodies. Thus, the population may be defined by the ability to maintain beating embryoid bodies upon prolonged in vitro culture. For example, in some aspects, a population of mesodermal precursor cells may comprises embryoid bodies wherein greater than about 10% of the embryoid bodies are beating after one month of culture. In still further aspects, such a population may comprise great that about 15%, 20%, 25%, 30%, 35% or more beating embryoid bodies after 4, 5, 6, 7, 8, 9, 10 or more weeks in tissue culture.

In still further aspects, a mesodermal precursor cell population may be defined by the ability to form tissue precursor structures. For example, in some aspects ERG overexpression directs differentiation of cells into cardiac and vascular tissue lineages that organize into tissue-like structures. As discussed above, in certain cases, such structures may be beating embryoid bodies. However, in further aspects, a mesodermal precursor cell population may be defined by the ability to form endothelial tissue structures such as endothelial cell tubes or more specifically hollow endothelial tubes. Furthermore, a mesodermal precursor cell population may be defined by its chemotactic properties. For example, in some aspects, wt-ERG expressing cells exhibit enhanced cell migration to other wt-ERG expressing cells as compared to controls cells that do not comprise a wt-ERG expression cassette. In still further aspects, a population of mesodermal precursor cells of the invention may be defined by their action potential characteristics. For instance, mesodermal progenitor (e.g., cardiac progenitor cells) cells may be may be defined by their electrophysiologic characteristics. For example, cells may be defined as having a hyperpolarized resting membrane potential, such as a potential of less than about −70 mV, (e.g., about −75 mV). Furthermore, in some aspects, a cell population of the invention may be defined as having shortened membrane action potentials (i.e., atrial or ventricular-like action potentials) relative to cells that lack an ERG expression vector and/or as a population of cells comprising homogeneous or uniform action potentials (see FIG. 2).

In still further aspects of the invention, mesodermal progenitor cells may be grown into more complex structured populations. For example, such populations may comprise beating embryoid bodies, myotubes, endothelial tubes or more advanced tissue-like structures comprising both myocardial and endothelial cell lineages. In some aspects, such a structured population may be defined as an anisotropic population of cells. For example, an anisotropic population may comprising self assembling rows or linear arrays of cells. Furthermore, in some aspects, cells of the invention may be arrayed in two dimensional sheets. Thus, in some aspects, there is provided a method for in vitro or ex vivo cardiac tissue engineering comprising growing mesodermal progenitor cells of the invention under conditions that are permissive for tissue formation. Thus, in some aspects, cells of the invention may be grown on or in a tissue formation matrix such as collagen or hydrogel (e.g., a MATRIGEL™ matrix) to mediate the formation of three dimensional tissue structures. Three dimension support matrices for use in such in vitro tissue growth has been described for example in U.S. Publn. Nos. 2006/0198827 and 2006/0210596, each incorporated herein by reference. In addition, in some aspect, monolayers and multilayers of tissues derived from the invention may be grown on temperature sensitive dishes to allow transfer of monolayers or multilayer to the surface of the heart. Thus, in some aspects, the invention provides methods for growing sections of tissue, such as cardiac tissue, in vitro for later transplantation into in vivo cardiac tissue.

In some further aspects, mesodermal progenitor cells of the invention may be treated with MEK1/ERG2 pathway agonists or antagonists. For example, in some cases MEK1 may be inhibited using small molecule antagonists such as PD98059 and U0126. Furthermore, in some aspects cells may be treated with TNF-α pathway antagonist or antagonists. For instance, SB203580 is a specific p38 MAPK inhibitor may be administered to cells. Sphingosine synthesis may is some cases, be blocked with an inhibitor of ceramidase, such as noleoyethanolamine. In still further aspects, A20, an inhibitor of NF-kappa B signaling pathway 70 may be applied to cells. By adding the foregoing additional compounds to the cells, their therapeutic effectiveness may be enhanced. For example, the tissue forming characteristics of the cells may be further stimulated or modulated by the addition of such additional compounds.

In still further aspects, mesodermal progenitor cells of the invention may be treated with VEGF or transformed with a VEGF expression vector. VEGF overexpression in bone marrow cells transplanted after myocardial infarction induced enhanced cardiomyogenesis and reduced infarct size (Haider & Ashraf, 2005; Janavel et al., 2006). Thus, in some aspects, mesodermal progenitor cells of the invention may be treated with VEGF or a molecule that enhances VEGF signaling prior to administration to an animal. Such methods may further enhance endothelial cell development or neovascularization both in vitro and in the milieu of damaged cardiac tissue (see, for instance, U.S. Patent Publ. 20050112104).

Thus, in still a further embodiments, there is provided a method for treating a patient with myocardial damage comprising (i) obtaining a population of mesodermal progenitor cells comprising an expression cassette comprising a wt-ERG gene under the control of a heterologous promoter and (ii) introducing an effective amount of the cells into the patient, thereby allowing the cells to integrate into the patients myocardial tissue. In some further aspects, a method for treating a patient with cardiac damage may comprise (i) obtaining a population of mesodermal progenitor cells of the invention, (ii) allowing the cells to grow in vitro to form a structured population of cells (e.g., as described supra) and (iii) introducing an effective amount of the mesodermal progenitor cells into the patient, thereby allowing the cells to integrate into the patients myocardial tissue. Methods of the invention may be used to treat a variety of cardiac damage. For example, cardiac tissue damaged by physical trauma, drug toxicity, pathogen infection (e.g., cardiotropic viral infection) or hypoxia may be treated by methods of the invention. Thus, in some aspects a patient suffering from myocardial infarction may be treated as described herein.

In still further aspects of the invention, cells for use in the methods of the invention may be derived from embryonic stem cells, cord blood cells, cord mesenchymal stem cells, cardiac stem cells or bone marrow as described supra. Thus, in some aspects, the cells may be defined comprising the same or similar markers for immunological compatibility (e.g., HLA type) as the patient being treated. In this aspect, the need for supportive immunosuppressive therapy may be reduced. In yet further aspects, cells for use in the invention may be derived from pluripotent cells from the patient such as cord blood or mesenchymal cells (e.g., frozen after birth) or bone marrow cells extracted from the patient.

In some aspects, methods of the invention may involve administering mesodermal progenitor cells or cells derived there from to a patient locally or systemically. For example, in some aspects, cells may be directly grafted or injected into the cardiac tissue of the patient. Thus, in some cases, cells may be administered directly to one or more sites of cardiac damage. For instance, cells of the invention may be used in conjunction with surgical repair of damaged heart tissue. In certain further embodiments, the cells may be administered systemically such as intravenously, intraarterially or intraperitoneally. Thus, in certain aspects, cells of the invention may be administered at a distant site from the heart but accumulate in damaged cardiac tissue. In some aspects, a factor may be administered with cells or after cell administration that enhances cell “homing” to the sick heart. Homing as used herein refers to the total number or rate at that wt-ERG cells accumulate site of interest (e.g., a cardiac lesion).

In still further aspects, the invention concerns methods for administering to a patient a structured population of mesodermal progenitor cells. For example, cells of the invention may be cultured in vitro as described supra and thereby allowed form cell sheets or tissue-like structures. Thus, in some instances, the invention concerns administering to a patient an anisotropic population of mesodermal progenitor cells. For example, a structured populations of cells may be directly grafted into a patient's cardiac tissue. Methods for such cell engraftment have been described for instance in PCT Appln. WO 06/080434. Thus, in still further aspects cells for the invention may be grown in vitro on a scaffolding or matrix to form a structured population of cells (e.g., a tissue-like population) prior to being administered to a patient.

Embodiments discussed in the context of a methods and/or composition of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: K⁺ channel current (IKr) features in WT (cell not overexpressing HERG) and HERG overexpressing ES cells. The IKr is assessed for WT (FIG. 1A) and HERG overexpressing ES cells (FIG. 1B). FIG. 1C, shows the action potential duration of CMDSC at 90% repolarization (APD₉₀) for WT and HERG overexpressing myocytes. FIG. 1D, show the resting membrane potential (RMP) in mV for the WT versus HERG overexpressing cardiomyocytes derived from stem cells. In each case error bar indicates standard deviation from the mean.

FIG. 2: Action potentials (AP) in cardiac myocytes. Cells derived from wild-type stem cells (CMDSC) manifest a spectrum of phenotypes whereas a homogeneous AP phenotype is seen in HERG OX CMDSC. Features include a short APD and hyperpolarization of RMP.

FIGS. 3A-D: The effect of HERG overexpression on dofetilide (DOF) responsiveness. HERG overexpression creates action potential which are responsive to dofetilide. AP is shown with-out DOF (FIG. 3A), or after 1 minute (FIG. 3B), 2 minute (FIG. 3C), or 3 minute treatments (FIG. 3D).

FIG. 4: Overexpression of HERG prolongs the longevity of beating embryoid bodies (filled circles) as compared to WT R1 cells. The percent of beating bodies in culture is graphed a function of time (d=days, w=weeks).

FIGS. 5A-C: Morphological differences between HERG OX cells versus control cells. Overexpression HERG mediates self assembly of a central beating mass with long linear extensions radiating from it (FIGS. 5A, B). No such extensions are generated in control cells (FIG. 5C).

FIGS. 6A-D: CMDSCs overexpressing HERG manifest self assembling tissue-like features versus amorphous mass formed by R1 (WT) cells. The figure shows α-actinin staining in the HERG overexpressing embryoid bodies (FIGS. 6C, D) compared to wild-type (FIGS. 6A-B) 2 days (FIG. 6A, C) or 10 days (FIGS. 6B, D) of culture.

FIGS. 7A-D: HERG OX cell populations comprise cells that have endothelial cell characteristics. Staining for the endothelial cell marker CD31 demonstrates that HERG OX cell populations (FIGS. 7B, D) have more CD31 cells and that cells form complex tube structures relative to WT cells (FIGS. 7A, C).

FIGS. 8A-D: HERG OX cell populations form hollow endothelial tubes. CD31 staining of vascular-like tube development HERG OX embryoid body cells cultured on OP9 feeders for 2 (FIG. 8C) or 10 days (FIG. 8D). Vascular-like tube development is absent from wild-type control cells after 2 (FIG. 8A) or 10 days (FIG. 8B) of culture.

FIGS. 9A-B: Chemotactic migration of HERG OX cells. FIG. 9A, a schematic diagram illustrating the chemotactic characteristics of HERG OX cells. FIG. 9B, a bar graph indicating the number of HERG OX cells that migrate towards other HERG OX cells or WT cells (as indicated).

FIG. 10: Transplantation of control embryonic stem cells and bone marrow cells moderately increases the survival of calcinurin overexpressing mice. Y-axis indicated the proportion of surviving animals, x-axis indicates survival time in weeks.

FIG. 11: Gross appearance of cells derived from adult cardiac tissue biopsies. Cells exposed to hERG overexpressing adenovirus.

FIG. 12: Sarcomeric actinin staining in the cells derived from adult cardiac tissue biopsies. Cells exposed to the hERG overexpressing adenovirus.

FIG. 13: α-actin staining in cells derived from adult cardiac fat pad biopsies. Cells exposed to hERG overexpressing adenovirus.

### DESCRIPTION OF ILLUSTRATIVE EMBODIMENTS

Methods for treating cardiac cell damage are still in their infancy and thus far the most effective approach has been to prevent the damage form occurring in the first place by minimizing the amount of time cardiac cells are exposed to a hypoxic state during a heart attack. Nonetheless, even the best preventative measures can not prevent all cardiac damage and thus methods for repairing cardiac damage with-out heart replacement would be a major advance. Along these lines a number researchers are currently experimenting with therapies that comprise administering pluripotent cell populations to a patient in the hope that such cells may differentiate into normal cardiac tissue to replace damage tissue regions (Wollert et al., 2004). Recently, there has been some success with injecting patients with hone marrow cells in order to achieve such tissue repair (Schachinger et al., 2006). However, even these therapies fail to provide the concentrated population of cardiac precursor cells that would be required for a therapy that would be effective as a standard treatment for myocardial infarction.

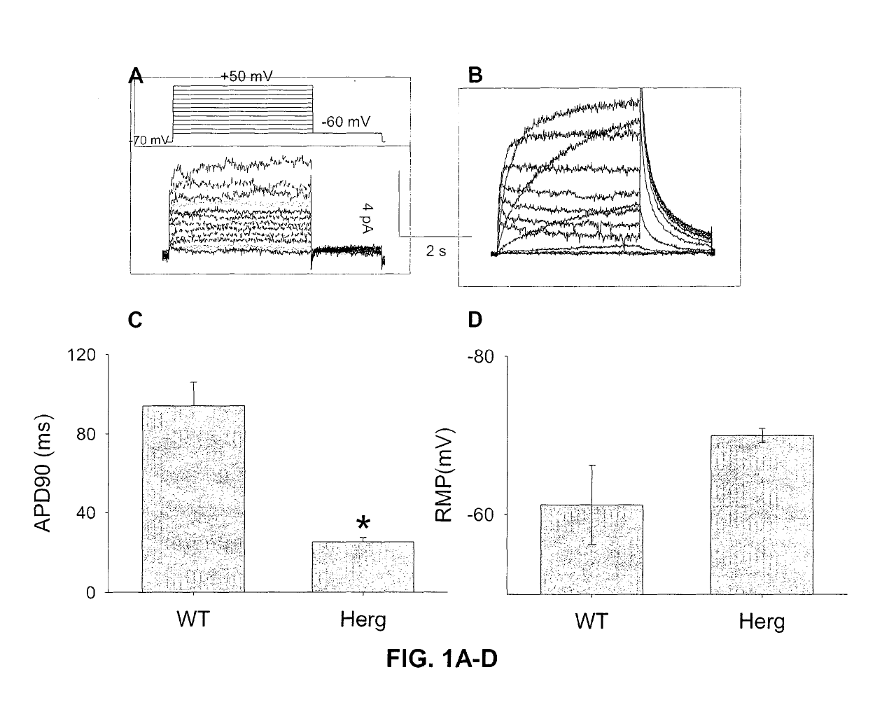

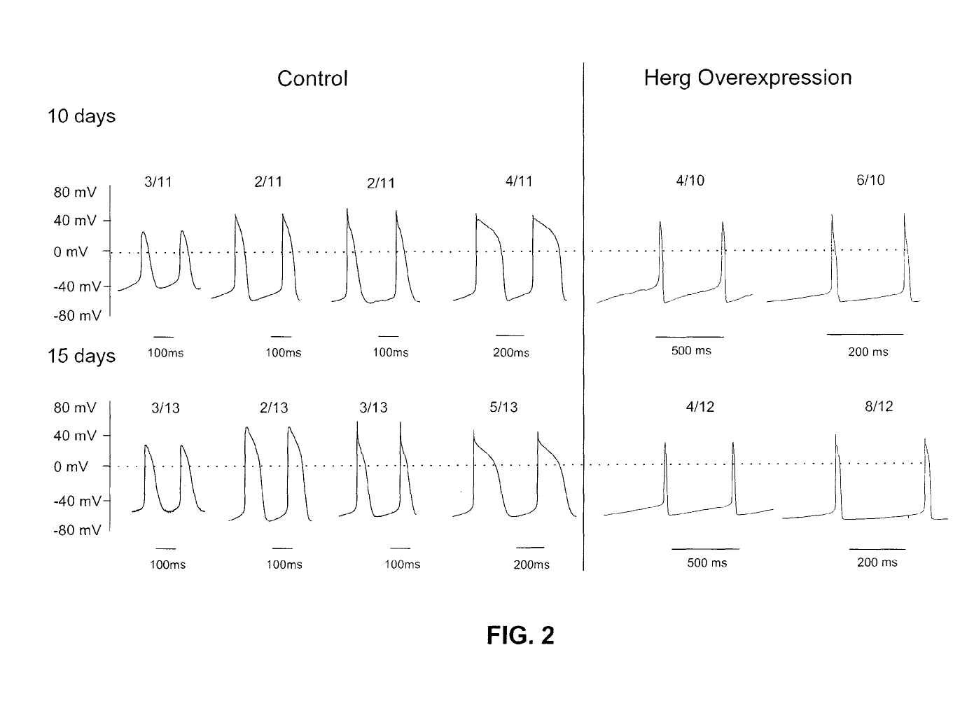

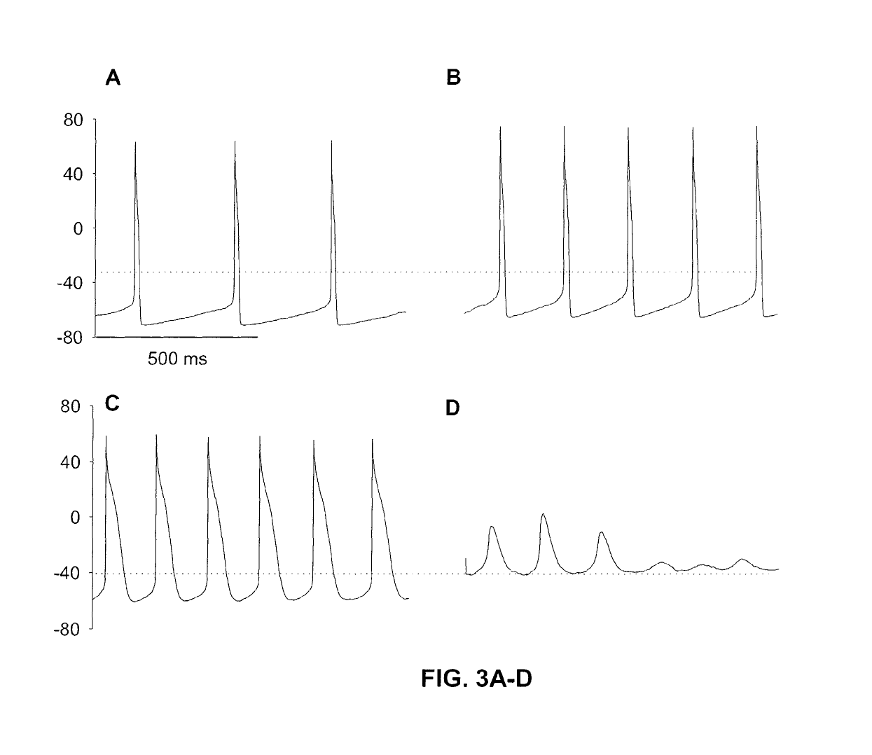

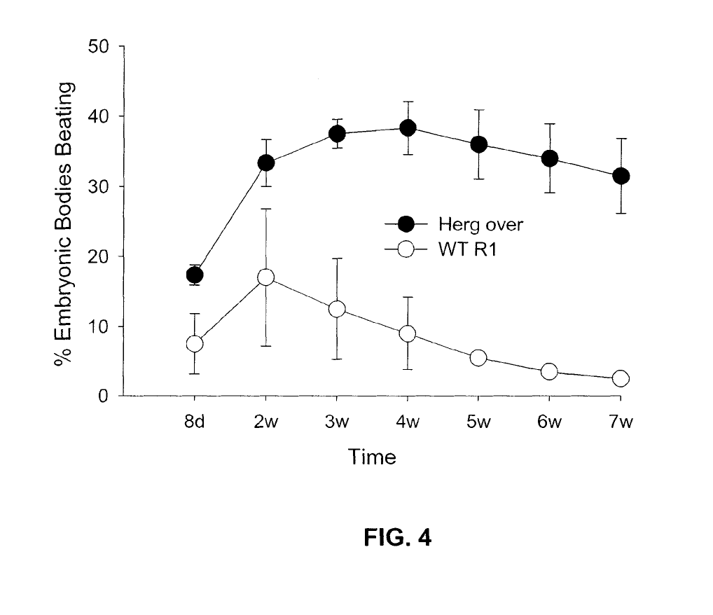

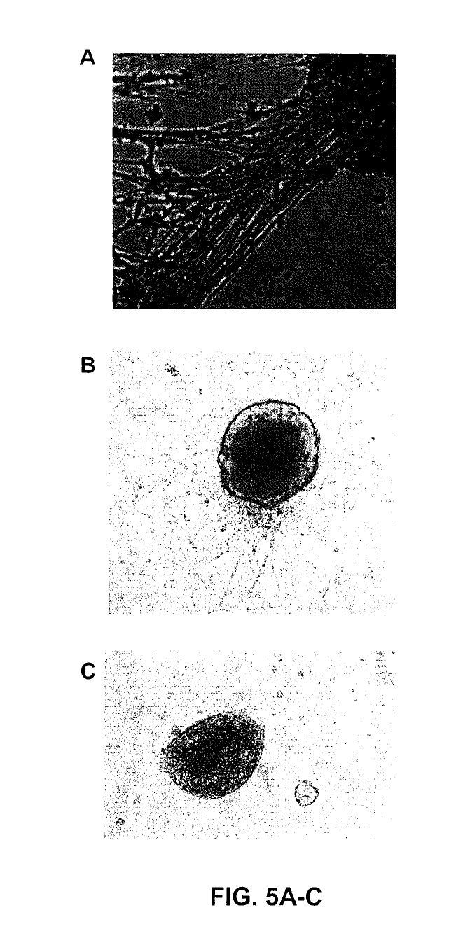

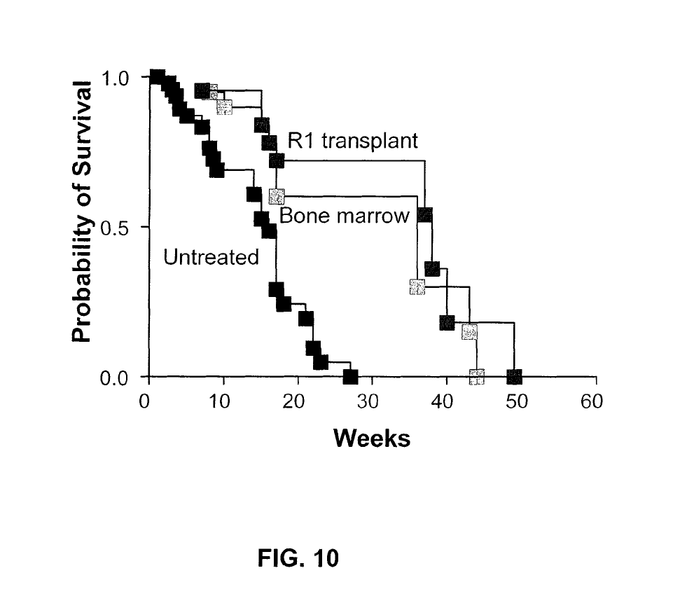

Studies outlined in the instant application represent a major advance in the development of mesodermal progenitor cells. Studies herein demonstrate that by expressing wt-ERG in pluripotent stem cells a population of mesodermal precursor cells with the ability to organize into self-assembling tissue-like structures can be generated. Moreover, overexpression of HERG directs differentiation of precursor cells into cardiac and vascular lineages. Cells expressing the wt-ERG exhibit action potentials that more closely represent the potentials of true myocardiocytes having a resting potential of about −78 mV (FIGS. 1A-D) and action potential phenotypes that are consistent across the cell population (FIG. 2). Furthermore, these cells gain other characteristics of normal myocardial tissue such as sensitivity to dofetilide (FIGS. 3A-D), prolonged longevity of beating embryoid bodies in the population (FIG. 4), and expression of myosin heavy chain. Furthermore, the cells population also cells that express endothelial markers. Importantly, HERG overexpressing cells form a mixed cell population with the ability to form tissue like structures such a myotubules and endothelial tubes even in the in vitro culture environment (FIGS. 5A-8D). This is of particular relevance since it suggests that the mesodermal progenitor population may be able to form organized tissue structures in vivo, which would be a requirement of effective tissue damage repair. Additionally, the wt-ERG expressing cells have enhanced chemotactic properties (FIG. 10), and are found to preferentially migrate toward one another further exemplifying their ability to organize tissue like formations. Finally, despite reports that ERG may have oncogenic activity, mice injected with cells comprising a wt-ERG expression cassette did not develop tumors. Thus, these studies indicate the wt-ERG expressing mesodermal precursor cells may be ideal therapeutic cells for the treatment of cardiac damage.

The invention provides new methods for generating mesodermal projector cells from a pluripotent cell population and further methods for using these cells to treat patients with cardiac damage. These methods a significant advance over previous methods that utilized pluripotent cell populations for treatment compositions. First, the cells of the invention comprise a cell population that is further directed towards a cardiac tissue differentiation path by expression of wt-ERG. Thus, wt-ERG expressing cells provide a more concentrated population of mesodermal precursor cells than was previously available. Additionally, these cells are able to organize into tissue-like structures, underscoring their use for in vivo tissue repair. Specifically, the cell population provided comprise both myocardiocyte and endothelial cell precursors thus providing the ability to not only repair damaged muscle tissue but also to form vascular networks needed to support the new tissue. Furthermore, the wt-ERG expressing cells have enhanced chemotactic proprieties and thus, when administered to a patient, may be able to home-in on damaged tissue and recruit additional wt-ERG mesodermal progenitor cells to the site of damage. Thus, the cells of the instant invention provide powerful new tools for treating cardiac tissue damage.

### I. NUCLEIC ACID EXPRESSION SYSTEMS

1. Vectors

The term “vector” is used to refer to a carrier nucleic acid molecule into which a nucleic acid sequence such as wt-ERG expression cassette can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Some vectors contemplated for use in the current invention include plasmids, cosmids, viruses (bacteriophage and animal viruses), and episomes (e.g., the EBV episome). One of skill in the art would be well equipped to construct a vector through standard recombinant techniques (see, for example, Maniatis et al., 1988 and Ausubel et al., 1994).

The term “expression vector” or “expression cassette” refer to any type of genetic construct comprising a nucleic acid coding for a RNA capable of being transcribed. In some cases, RNA molecules are then translated into a protein, polypeptide, or peptide. Thus, a wt-ERG expression cassette of the invention comprises all of the necessary elements for wt-ERG RNA transcription and translation.

a. Promoters and Enhancers

A “promoter” is a control sequence that is a region of a nucleic acid sequence at which initiation and rate of transcription are controlled. A promoter may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. Thus, a “heterologous promoter” may be any promoter other than the promoter that naturally controls transcription of a gene. Thus, in certain aspects, a heterologous promoter for use herein is any promoter other than an ERG promoter.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 by upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 by apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e. containing different elements of different transcriptional regulatory regions, and/or mutations that alter expression. For example, promoters that are most commonly used in recombinant DNA construction include the (3 lactamase (penicillinase), lactose and tryptophan (tip) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, www.epd.isb-sib.ch) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler |
| | et al., 1987; Weinberger et al., 1984; Kiledjian |
| | et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al, 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson |
| | et al., 1989 |
| Prealbumin (Transthyretin) | Costa et al., 1988 |
| Elastase 1 | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester | Palmiter et al., 1982; |
| | (TFA) | Haslinger et al., 1985; |
| | Heavy metals | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse mammary | Glucocor- | Huang et al., 1981; Lee |
| tumor virus) | ticoids | et al., 1981; Majors et al., |
| | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 | Taylor et al., 1989, 1990a, |
| | Large T | 1990b |
| | Antigen | |
| Proliferin | Phorbol Ester- | Mordacq et al., 1989 |
| | TPA | |
| Tumor Necrosis Factor α | PMA | Hensel et al., 1989 |
| Thyroid Stimulating | Thyroid | Chatterjee et al., 1989 |
| Hormone α Gene | Hormone | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (X1) collagen (Tsumaki at al., 1998), D1A dopamine receptor gene (Lee at al., 1997), insulin-like growth factor II (Wu at al., 1997), and human platelet endothelial cell adhesion molecule-1 (Almendro at al., 1996).

b. Translation Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference).

e. Polyadenylation Signals

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Thus, in expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

f. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

g. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, blastacidin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

h. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEMTM 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with (3 galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

i. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Thus, a wt-ERG expression cassette of the present invention may be delivered into a pluripotent cell using a viral vector. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use according to the present invention as it has a high frequency of integration into the host cell genome, either a specific locus on chromosome 19 or randomly, depending upon the inclusion of the AAV Rep gene in the vector. AAV also has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as a wt-ERG delivery vectors also due to their ability to integrate their genes into the host genome, transferring a large amount of foreign genetic material, infecting a broad spectrum of species and cell types and of being packaged in special cell lines (Miller, 1992).

In order to construct a retroviral vector comprising a wt-ERG expression cassette, a wt-ERG gene (and optionally a promoter) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of cell comprising sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989; Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985), and any combination of such methods. Through the application of techniques such as these, organelle(s), cell(s), tissue(s) or organism(s) may be stably or transiently transformed.

a. Ex Vivo Transformation

Methods for transfecting cell populations removed from an organism in an ex vivo setting are known to those of skill in the art. For example, canine endothelial cells have been genetically altered by retroviral gene transfer in vitro and transplanted into a canine (Wilson et al., 1989). In another example, yucatan minipig endothelial cells were transfected by retrovirus in vitro and transplated into an artery using a double-balloon catheter (Nabel et al., 1989). Thus, it is contemplated that cells or tissues may be removed and transfected ex vivo using the nucleic acids of the present invention. In particular aspects, the transplanted cells or tissues may be placed into an organism as described further herein. In certain aspects, wt-ERG remains expressed in the transplanted cells or tissues.

b. Electroporation

In certain embodiments of the present invention, a nucleic acid is introduced into an organelle, a cell, a tissue or an organism via electroporation. Electroporation involves the exposure of a suspension of cells and DNA to a high voltage electric discharge. In some variants of this method, certain cell wall degrading enzymes, such as pectin degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre B lymphocytes have been transfected with human kappa immunoglobulin genes (Potter et al., 1984), and rat hepatocytes have been transfected with the chloramphenicol acetyltransferase gene (Tur Kaspa et al., 1986) in this manner.

c. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV 1, BHK, NIH3T3 and HeLa cells were transfected with a neomycin marker gene (Chen and Okayama, 1987), and rat hepatocytes were transfected with a variety of marker genes (Rippe et al., 1990).

d. DEAE Dextran

In another embodiment, a nucleic acid is delivered into a cell using DEAE dextran followed by polyethylene glycol. In this manner, reporter plasmids were introduced into mouse myeloma and erythroleukemia cells (Gopal, 1985).

e. Sonication Loading

Additional embodiments of the present invention include the introduction of a nucleic acid by direct sonic loading. LTK fibroblasts have been transfected with the thymidine kinase gene by sonication loading (Fechheimer et al., 1987).

f. Liposome Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome mediated nucleic acid delivery and expression of foreign DNA in vitro has been very successful (Nicolau and Sene, 1982; Fraley et al., 1979; Nicolau et al., 1987). The feasibility of liposome mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome encapsulated DNA (Kaneda et al., 1989). In other embodiments, a liposome may be complexed or employed in conjunction with nuclear non histone chromosomal proteins (HMG 1) (Kato et al., 1991). In yet further embodiments, a liposome may be complexed or employed in conjunction with both HVJ and HMG 1. In other embodiments, a delivery vehicle may comprise a ligand and a liposome.

g. Receptor Mediated Transfection

Still further, a nucleic acid may be delivered to a target cell via receptor mediated delivery vehicles. These take advantage of the selective uptake of macromolecules by receptor mediated endocytosis that will be occurring in a target cell. In view of the cell type specific distribution of various receptors, this delivery method adds another degree of specificity to the present invention.

Certain receptor mediated gene targeting vehicles comprise a cell receptor specific ligand and a nucleic acid binding agent. Others comprise a cell receptor specific ligand to which the nucleic acid to be delivered has been operatively attached. Several ligands have been used for receptor mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell specific binding. For example, lactosyl ceramide, a galactose terminal asialganglioside, have been incorporated into liposomes and observed an increase in the uptake of the insulin gene by hepatocytes (Nicolau et al., 1987). It is contemplated that the tissue specific transforming constructs of the present invention can be specifically delivered into a target cell in a similar manner.

h. Microprojectile Bombardment

Microprojectile bombardment techniques can be used to introduce a nucleic acid into at least one, organelle, cell, tissue or organism (U.S. Pat. Nos. 5,550,318, 5,538,880, 5,610,042; and PCT Application WO 94/09699; each of which is incorporated herein by reference). This method depends on the ability to accelerate DNA coated microprojectiles to a high velocity allowing them to pierce cell membranes and enter cells without killing them (Klein et al., 1987). There are a wide variety of microprojectile bombardment techniques known in the art, many of which are applicable to the invention.

For microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

3. Expression

It is contemplated that the wt-ERG in cells of the invention may be “overexpressed,” i.e., expressed in increased levels relative to its natural expression in cells. Such overexpression may be assessed by a variety of methods, including radio labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

### II. CELL CULTURE AND ISOLATION

Methods for the culture human stem cells are known in the art (U.S. Pat. No. 6,200,806). More specific methods for the culture and differentiation of mesodermal progenitor cells have also been described, for example see Tang et al. (2004) and U.S. Pat. No. 5,486,359. For example, in some embodiments of the invention, cells may be cultured in a serum-free media. This aspect may reduce the chance for viral or bacterial contamination of the cells arising from serum preparations of biological origin. Methods for serum-free culture of mammalian mesenchymal cells, such as culture on 3-dimensional scaffolds have been previously described in U.S. Patent Publ. 2005/0265980.

Mesoderm progenitor cells of the invention may be isolated from a variety of tissues. Bone marrow cells have previously shown some therapeutic efficacy in cardiac tissue repair. Thus, in certain cases, isolated bone marrow cells maybe used in methods of the invention. Methods for isolating bone marrow cells have been previously described and the culture of these cells is well known in the art. Furthermore, methods for enriching populations of bone marrow cells to favor mesenchymal cells have also been described. For example, bone marrow cells may be cultured in an atrial conditioned media to induce a mesenchymal lineage (PCT Appln. WO 05/054448). Other methods for deriving mesodermal cells from bone marrow have been described in Horwitz et al. (1999) and Koc et al. (2000). In still further aspects of the invention other adult tissues may be used as a source of progenitor cells of the invention. For example, adult cardiac tissue comprises populations of cells that are capable of mesenchymal differentiation (Kattman et al., 2006). Thus, in some aspects, cells obtain via cardiac biopsy may be used in the instant methods. Methods have been previously reported to obtain cardiac-committed progenitor cells (Smith et al., 2007; Messina et al., 2004). These cells are characterized by expression of c-Kit, CD34, and CD31, optionally further including CD90, CD105 and sca-1, optionally further lacking MDR1, CD133 and CD45.

Furthermore, methods for deriving mesenchymal cells from adipose tissues have also been described (see PCT Applns. WO 06/080434 and WO 06/017320). Methods for extracting mesenchymal stem cells from peripheral blood are also known. For instance, an enhanced number of such stem cells may be extracted from the blood of individuals that have been administered certain growth factors such as G-CSF and/or GM-CSF (U.S. Pat. No. 6,261,549).

In some cases, progenitor cells for use in the invention may be derived for fetal or uterine tissues. For example, menstrual blood, cord blood or fetal appendages may be used as a source for cells. Such cells have been shown to differentiate into mesenchymal lineages, for example when co-cultured with cardiac cells (PCT Appln. WO 06/078034). Thus, to in some aspects, umbilical cord mesenchymal cells may be used according to the methods of the invention. Methods for isolating and characterizing cord mesenchymal cells have been previously described (Bieback et al., 2004).

### III. COMBINATION THERAPIES

In some aspects of the invention subjects with cardiac damage are treated with mesodermal progenitor cells expressing wt-ERG. It is also contemplated that other therapeutic regimens may be used in combination or in conjunction these methods.

1. Anti-Inflammatory Agents

In certain aspect a patient treated according to the invention may be administered a immunosuppressive or anti-inflammatory drug. For example, the drug may be administered in order to increase the chances that mesodermal progenitor cells used to treat cardiac damage are not destroyed by the patients immune system. For instance a patient may be treated with mycophenolate mofetil (MMF), a rapamycin or rapamycin analog. A wide variety of other anti-inflammatory agents are known to one of skill in the art. Most commonly used are the nonsteroidal anti-inflammatory agents (NSAIDs) which work by inhibiting the production of prostaglandins. Non-limiting examples include, ibuprofen, ketoprofen, piroxicam, naproxen, naproxen sodium, sulindac, aspirin, choline subsalicylate, diflunisal, oxaprozin, diclofenac sodium delayed release, diclofenac potassium immediate release, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate. Another group of anti-inflammatory agents comprise steroid based potent anti-inflammatory agents, for example, the corticosteroids which are exemplified by dexamethason, hydrocortisone, methylprednisolone, prednisone, and triamcinolone as non-limiting examples. Several of these anti-inflammatory agents are available under well known brand names, for example, the NSAIDs comprising ibuprofen include Advil, Motrin IB, Nuprin; NSAIDs comprising acetaminophens include Tylenol; NSAIDs comprising naproxen include Aleve.

2. Antiarrhythmic Therapies

For example, in some aspects antiarrhythmic drugs may be used in individuals prior to or after administration of cells of the invention. For example, Dofetilide (Tikosyn®), a class III antiarrhythmic agent is used for the conversion to and maintenance of normal sinus rhythm in patients with highly symptomatic flutter and cells of the invention have been shown to be sensitive to this drug. Dofetilide prolongs both atrial and ventricular repolarization, and therefore increases the refractory period within the cardiac muscle. Other class III antiarrhythmic agents comprise other antiarrhythmic properties. For instance, Sotalol (a β-blocker) and amiodarone decrease AV nodal conduction.

3. β-Blockers and ACE Inhibitors

One primary for of therapy of patients having suffered myocardial infarction is β-blocker therapy and/or angiotensin converting enzyme (ACE) inhibitor therapy. These therapies are typically shortly after acute presentation and is continued indefinitely. Thus, patients treated according to the invention may additionally be treated with β-blocker therapy. Some beta blocker drugs that may be used in conjunction with the instant invention include but are not limiter to, Metoprolol, Atenolol, Esmolol, Betaxolol, Bisoprolol, Acebutolol and Propranolol. Furthermore, patients administered cells of the invention may also be administered an ACE inhibitor.

4. Surgical Approaches

One primary therapeutic approach to myocardial infarction is surgical revascularization of the effected area. Thus, in certain aspects such surgical therapy may be combine with the cell administration methods of the invention. Thus, in some aspects, surgical revascularization of tissue may be aided by also providing cells of the invention at the site of the infarction to further revascularize the effected region and repair the damaged myocardial tissue.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Characteristics of Cells Overexpressing HERG

To establish cardiac myocytes derived from embryonic stem cells (CMDSC) which stably overexpress hERG, R1 ESC were electroporated with a DNA construct comprising wt HERG gene and green fluorescent protein (GFP) reporter gene under the control of a CMV-IE chicken β-actin promoter. The characteristics of the HERG overexpressing CMDSC were then determined. As shown in FIGS. 1A-D and FIG. 2, APD₉₀ and RMP were significantly altered in the HERG overexpressing stein cells. The HERG expressing cells exhibited hyperpolarization of resting potential to −78 mV and have significantly more homogeneous action potential profiles relative to untransformed cells (FIG. 2). Furthermore, as shown in FIGS. 3A-D the HERG overexpressing cells are sensitive to the effects of dofetilide.

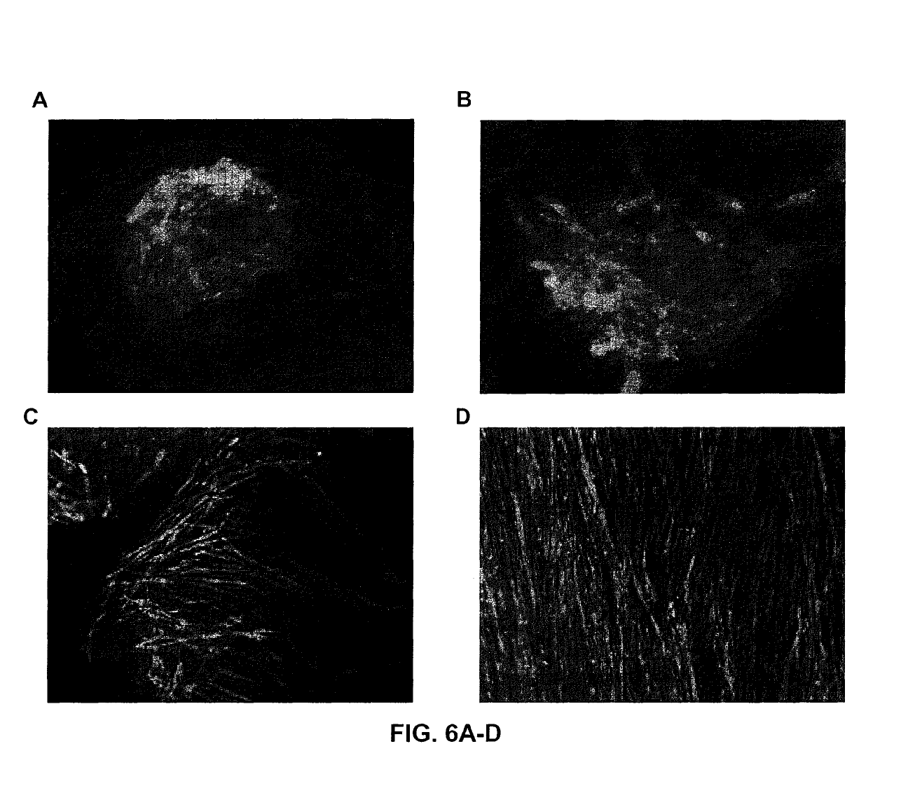

Overexpression of HERG also improves the longevity of beating embryoid bodies (FIG. 4). Even after 7 weeks of culture, over 30% of embryoid bodies had a spontaneous heating phenotype as compared to less than 10% of untransformed cells. HERG expression also alters the morphology of embryoid bodies overexpressing hERG. As shown in FIGS. 5A-C, HERG cells form a central beating mass with linear extensions. The linear extensions stain with MF-20 (myosin heavy chain), indicating increased differentiation of cells into myocytes. The HERG cells (FIGS. 6C,D) rapidly begin assembling tissue-like (anisotropic) architecture as compared to the amorphous mass that is formed by control cells (FIGS. 6A,B). This is important because anisotropic architecture is a characteristic of a tissue rather than a cell.

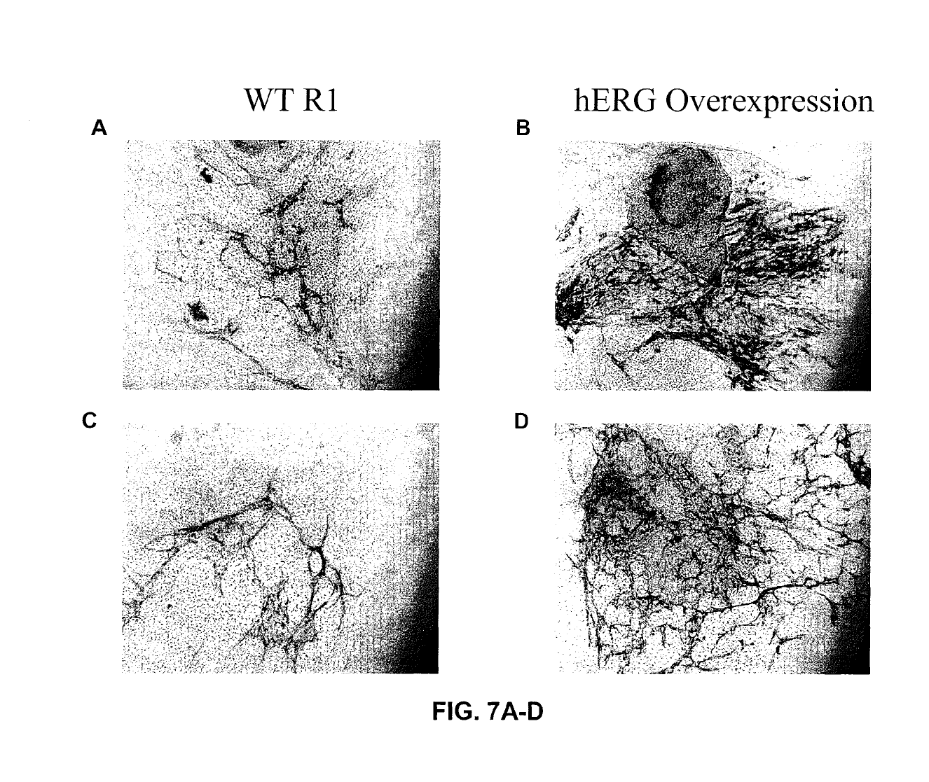

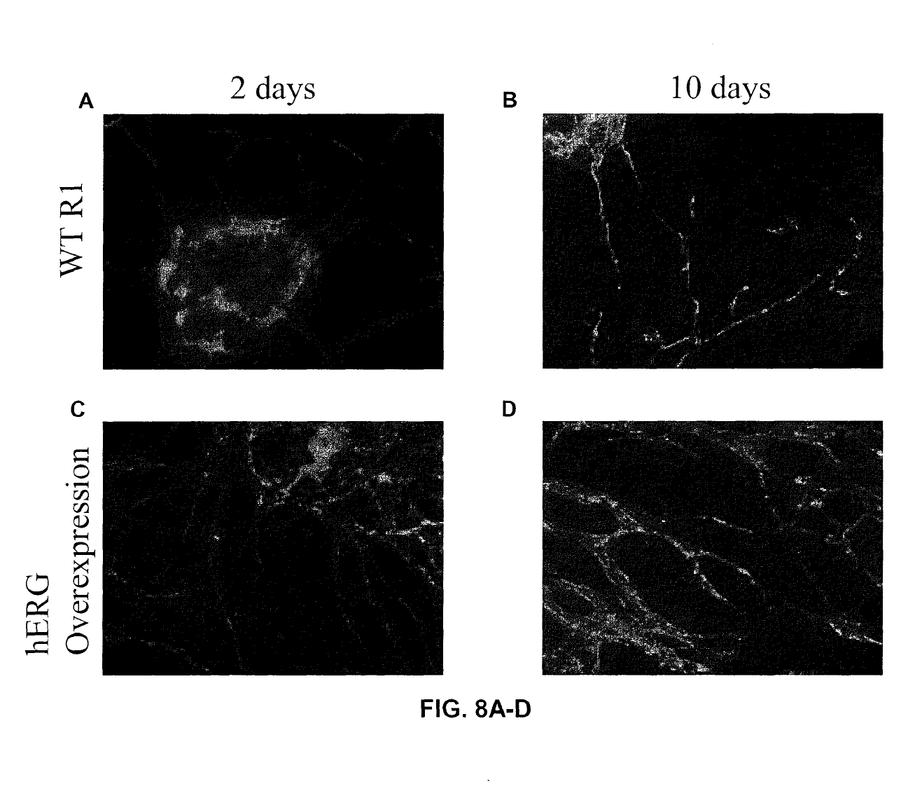

Furthermore, overexpression of HERG in embryoid bodies derived from stem cells generates an architecture of CD-31 staining cells (endothelial lineage indicator) spreading over the collagen coated culture dish. (FIGS. 7B,D). When cells are grown on a OP-9 feeder layer, the organized CD-31 staining structures grossly appear to be self assembling into early vascular-like tubes (FIG. 8D). This spreading is exaggerated compared to WT (FIGS. 7A,C). Self-assembly into vascular tubes is novel.

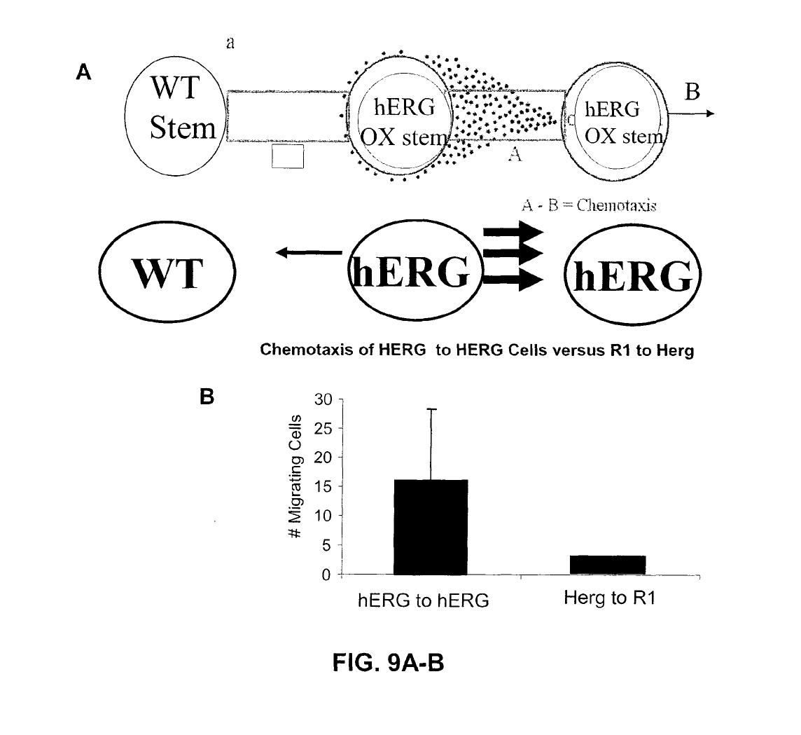

HERG cells were also assessed to determine whether paracrine chemotactic factors direct the migration of stem cells and CMDSC, comparing R1-control versus HERG overexpression cells. An under agarose assay was developed by the Kubes lab was used (Heit et al., 2002; Heit & Kubes, 2003). Briefly, pairs of wells are filled with a stem cell or a chemoattractant. The chemoattractant can be a chemical or a cell locally secreting a chemoattractant (e.g., a HERG OX cell). The method has required minor modification to assess chemotaxis of stem cells rather than neutrophils, which are one of the most motile cells in the body. Mobility is recorded using a video camera attached to a ZEISS Axiovert 135 microscope. The under agarose assay allows quantification of both random migration (chemokinesis) and directional migration (chemotaxis). To determine if the migration is directional, two target areas are analyzed: Target A is the segment between the stem cell well and the chemoattractant well. Target B is the same size as Target but extending away from the stem cell well to a point 180 degrees opposite to the chemoattractant well (FIG. 9A). The number of chemotaxing cells is determined by subtracting the number of migrating cells in target B from the number of cells in area A. Interestingly, HERG OX cells demonstrate increased cheomotaxis as compared to control R1 cells. A much greater number of HERG OX cells migrate other HERG cells than to control R1 cells. Thus, phenotype as well may be very important in complex structure formation.

### Example 2

### In Vivo Introduction Pluripotent Cells

The calcineurin-overexpression mouse model (CN) has been evaluated both by echocardiography and electrophysiology. Using this model the capacity of bone marrow transplants or R1 ESC to improve longevity and echocardiographic indices of cardiac performance of mice were evaluated (FIG. 9B). Mice overexpressing calcineurin (CN) seem ideal for these studies. For seven generations the CN mice have been inbred into the SV129 strain of mice to minimize any possible antigenic rejection of R1 derived stem cells. Importantly, 100% of calcineurin mice die due to arrhythmic cardiac death associated with end stage congestive heart failure and myocardial fibrosis. Thus, this mouse model has a pathologic substrate similar to that which induces arrhythmic death in humans. Studies transplanting R1 stem cells and bone marrow show a modest but significant improvement in longevity from 25 to 38 weeks. Importantly, improved echocardiographic metrics of myocardial performance following transplantation have also been demonstrated.

### Example 3

### HERG OX Cells do not Mediate Tumor Genesis In Vivo

To assess potential tumorogenesis of R1 versus HERG overexpressing cells both lines were transplanted in vivo. Four mice have been injected intravenously with HERG overexpression and R1 stem cells. At 3 months following transplantation, they were autopsied and no tumors were found at gross inspection or by random biopsy in either case.

### Example 4

### Isolation, Culture and Overexpression of the hERG Potassium Channel by Adenoviral Infection of Human Cells Derived from Adult Cardiac Muscle and Fat Pad Biopsies

Isolation and Culture. Human right atrial appendages and cardiac fat pad tissue was derived from biopsy specimens. This study was approved by the Univ. of Calgary human ethics committee and informed consent was obtained from each patient. Isolated cardiac/cardiac fat pad tissue were cut 1-2 mm pieces, washed with PBS for three times. The minced tissue was incubated with 1 mg/ml collagenase type II for 30-60 minutes with gentle agitation at 37° C. The digested mixture was then passed through a 100 μM filter to obtain cell suspensions. Cells were plated in 0.1% gelatin coated culture dishes growth medium consisted of Dulbecco's modified Eagle's medium (DMEM) supplemented with 10% fetal calf serum; 100 U/ml penicillin; 1 mM sodium pyruvate; 100 μM non-essential amino acids; and 55 μM 2-mercaptoethanol and 1000 U/ml leukemia inhibitory factor (LIF). Cultures were maintained in a humidified atmosphere with 5% CO, at 37° C. After 24 hr, the medium was replaced and non-adherent cells were remove. The medium was then changed twice weekly. Once 80-90% confluence had been reached, the cells were replated for expansion. This method is a minor modification of that which has been previously reported to obtain cardiac-committed progenitor cells (Smith et al., 2007; Messina et at, 2004).

hERG adenovirus infection. Recombinant adenovirus containing the cDNA for hERG were used for hERG overexpression in cells derived from the adult human biopsies. The dispersed cells were cultured on 0.1% gelatin-coated glass cover slips in 24-well culture plates with the above growth medium without LIF, after 90% confluence the cells were infected with herg virus or GFP alone virus (as control), after culture another 10 days, the cells were fixed with methanol (−20° C.) for 10 minutes, air dried and then were kept in 1% BSA for immunofluoresence analysis using anti-α-actinin or anti-α-actin antibodies.

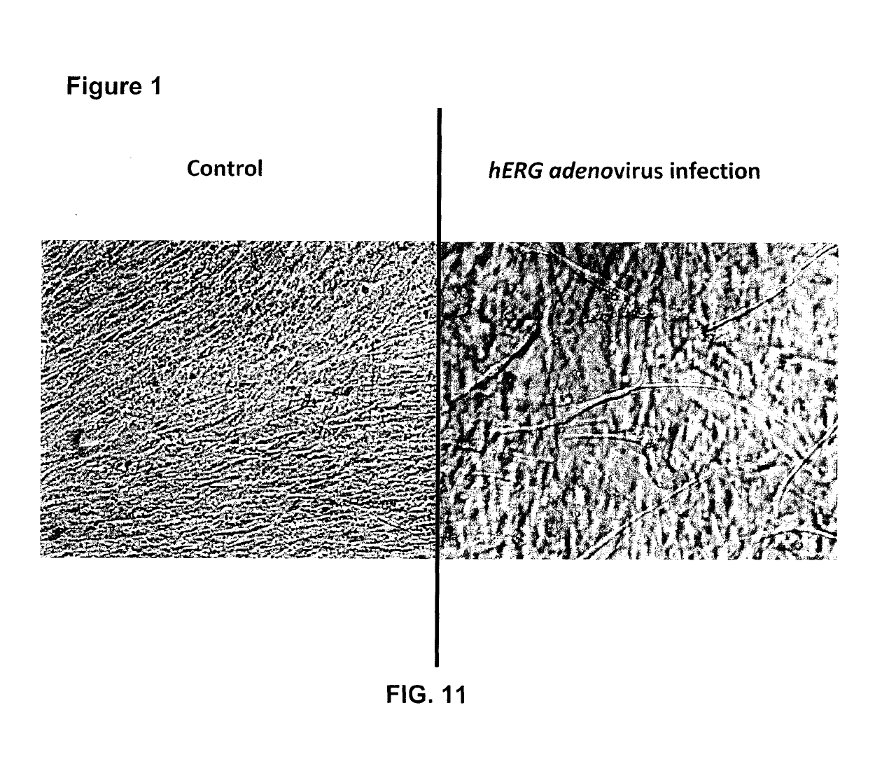

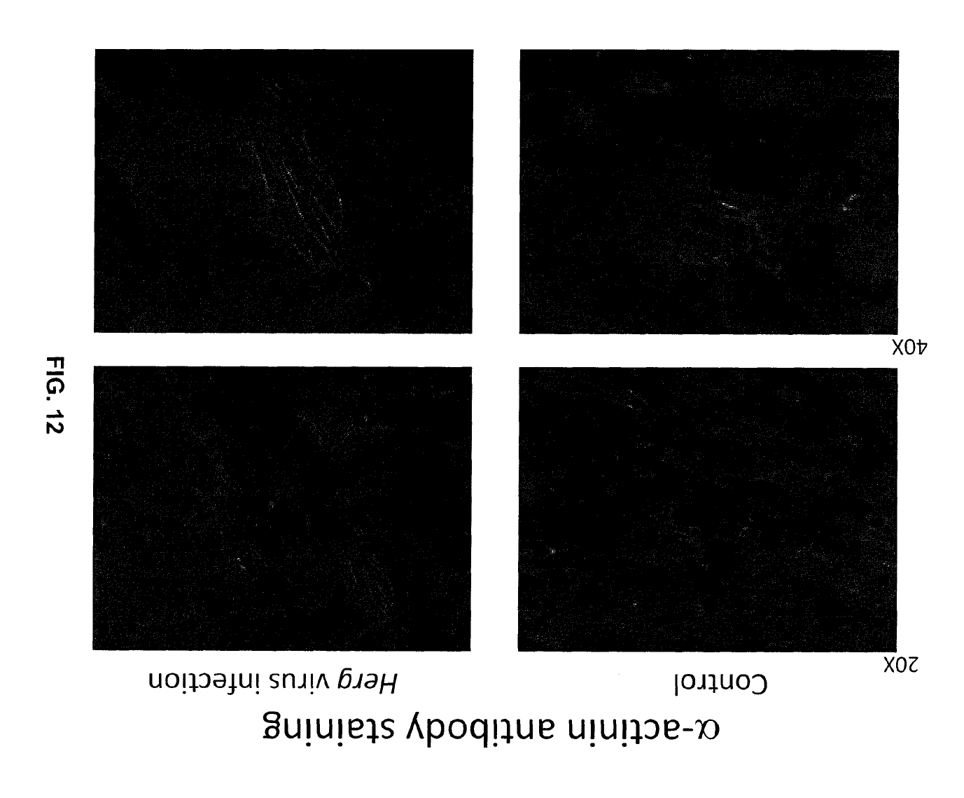

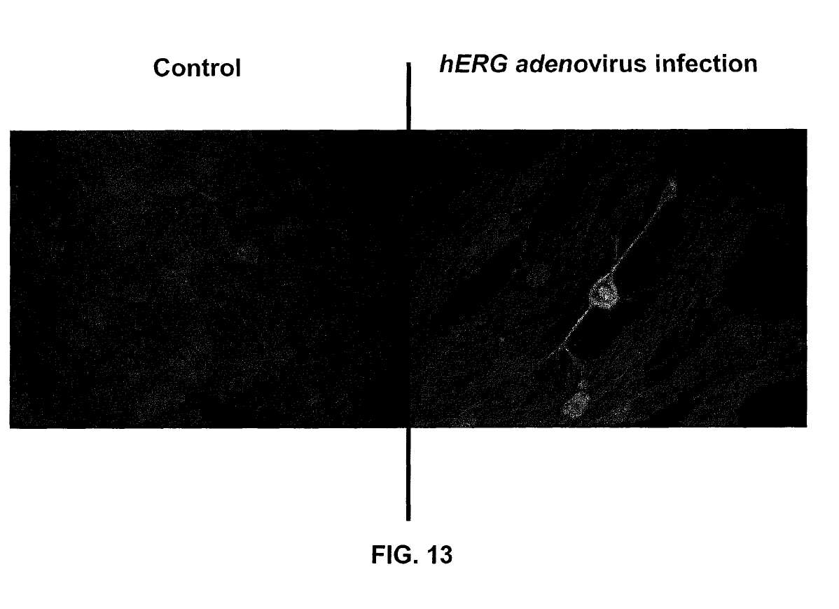

Results. FIG. 11 shows the gross appearance of cells derived from adult cardiac tissue biopses. Cells exposed to hERG overexpressing adenovirus manifest a subpopulation of cells that outgrow that have long linear extensions with anisotropic properties. FIG. 12 shows sarcomeric actinin staining in the cells derived from adult cardiac tissue biopsies. Cells exposed to the hERG overexpressing adenovirus had substantially greater sarcomeric organization (indicating muscle lineage) than in control cells. This reflects a greater likelihood of differentiating to cardiac myocytes. FIG. 13 shows α-actin staining in cells derived from adult cardiac fat pad biopsies. These cells, when exposed to hERG overexpressing adenovirus, manifest the development of a unique cell type that had feature of anisotropic growth. These data provide evidence of proof of concept. Overexpression of the hERG potassium channels in cells derived from adult human biopsies allows an alteration of the phenotype and apparent lineage of cells derived from those biopsies.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,202
U.S. Pat. No. 4,684,611
U.S. Pat. No. 4,797,368
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,384,253
U.S. Pat. No. 5,464,765
U.S. Pat. No. 5,486,359
U.S. Pat. No. 5,538,877
U.S. Pat. No. 5,538,880
U.S. Pat. No. 5,538,880
U.S. Pat. No. 5,550,318
U.S. Pat. No. 5,550,318
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,925,565
U.S. Pat. No. 5,928,906
U.S. Pat. No. 5,935,819
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 6,200,806
U.S. Pat. No. 6,261,549
U.S. Patent Publ. 2003000874
U.S. Patent Publ. 20050112104
U.S. Patent Publ. 20050265980
U.S. Patent Publ. 20060198827
U.S. Patent Publ. 20060210596
Almendro et al., J Immunol., 157(12):5411-21, 1996.
Angel et al., Cell, 49:729, 1987b.
Angel et al., Mol. Cell. Biol., 7:2256, 1987a.
Arcangeli et al J. Physiol. 489:455-471, 1995.
Arcangeli et al., Biochem Soc Trans., 32:826-827, 2004.
Arcangeli et al., Cell Adhes Commun. 4:369-385, 1996.
Arcangeli, Novartis Found Symp. 266: 225-234, 2005.
Atchison and Perry, Cell, 46:253, 1986.
Atchison and Perry, Cell, 48:121, 1987.
Ausubel et al., In: Current Protocols in Molecular Biology, John, Wiley & Sons, Inc, NY, 1994.
Baichwal and Sugden, In: Gene Transfer, Kucherlapati (Ed.), Plenum Press, NY, 117-148, 1986.
Banerji et al., Cell, 27:299, 1981.
Banerji et al., Cell, 35:729, 1983.
Berkhout et al., Cell, 59:273, 1989.
Bianchi et al., Biochem Biophys Res Commun., 210:823-829, 1995.
Bieback et al., Stem Cells, 22:625-634, 2004.
Blanar et al., EMBO J., 8:1139, 1989.
Blomer et al., J. Virol., 71(9):6641-6649, 1997.
Bodine and Ley, EMBO J., 6:2997, 1987.
Boshart et al., Cell, 41:521, 1985.
Bosze et al., EMBO J., 5:1615, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatterjee et al., Proc. Nat'l Acad. Sci. USA., 86:9114, 1989.
Chen and Okayama, Mol. Cell. Biol., 7(8):2745-2752, 1987.
Cherubini et al., Ann N Y Acad. Sci., 973:559-561, 2002.
Cherubini et al., Mol Biol Cell., 16:2972-2983, 2005.
Choi et al., Cell, 53:519, 1988.
Cocea, Biotechniques, 23(5):814-816, 1997.
Cohen et al., J. Cell. Physiol., 5:75, 1987.
Costa et al., Mol. Cell. Biol., 8:81, 1988.
Cotten et al., Proc. Natl. Acad. Sci. USA, 89(13):6094-6098, 1992.
Coupar et al., Gene, 68:1-10, 1988.
Cripe et al., EMBO J., 6:3745, 1987.
Crociani et al., J Biol Chem, 278:2947-2955, 2003.
Culotta and Hamer, Mol. Cell. Biol., 9:1376, 1989.
Curiel, Nat. Immun., 13(2-3):141-164, 1994.
Dandolo et al., J. Virology, 47:55, 1983.
de Villiers et al., Nature, 312:242, 1984.
Deschamps et al., Science, 230:1174, 1985.
Edbrooke et al., Mol. Cell. Biol., 9:1908, 1989.
Edlund et al., Science, 230:912, 1985.
EPO 0273085
Fechheimer, et al., Proc Natl. Acad. Sci. USA, 84:8463-8467, 1987,
Feng and Holland, Nature, 334:6178, 1988.
Firak and Subramanian, Mol. Cell. Biol., 6:3667, 1986.
Foecking and Hofstetter, Gene, 45:101, 1986.
Fraley et al., Proc. Natl. Acad. Sci. USA, 76:3348-3352, 1979.
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodbourn et al., Cell, 45:601, 1986.
Gopal, Mol. Cell. Biol., 5:1188-1190, 1985.
Graham and Van Der Eb, Virology, 52:456-467, 1973.
Greene et al., Immunology Today, 10:272, 1989.
Grosschedl and Baltimore, Cell, 41:885, 1985.
Grunhaus and Horwitz, Seminar in Virology, 3:237-252, 1992.
Haider & Ashraf, Cytotherapy, 7:74-75, 2005.
Harland and Weintraub, J. Cell Biol., 101(3):1094-1099, 1985.
Haslinger and Karin, Proc. Nat'l Acad. Sci. USA., 82:8572, 1985.
Hauber and Cullen, J. Virology, 62:673, 1988.
Heit & Kubes, Sci STKE., 2003:PL5, 2003.
Heit et al., J. Cell Biol., 159:91-102, 2002.
Hen et al., Nature, 321:249, 1986.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10:1959, 1990.
Hofmann et al., J Biol. Chem., 276: 4923-4931, 2001.
Holbrook et al., Virology, 157:211, 1987.
Horlick and Benfield, Mol. Cell. Biol., 9:2396, 1989.
Horwich at al. J. Virol., 64:642-650, 1990.
Horwitz et al., Nat. Med., 5:309-313, 1999.
Huang et al., Cell, 27:245, 1981.
Hug at al., Mol. Cell. Biol., 8(8):3065-79, 1988.
Hwang et al., Mol. Cell. Biol., 10:585, 1990.
Imagawa at al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Inouye and Inouye, Nucleic Acids Res., 13:3101-3109, 1985.
Jakobovits et al., Mol. Cell. Biol., 8:2555, 1988.
Jameel and Siddiqui, Mol. Cell. Biol., 6:710, 1986.
Janavel et al., Gene Ther., 13:1133-1142, 2006.
Jaynes at al., Mol. Cell. Biol., 8:62, 1988.
Johnson et al., Mol. Cell. Biol., 9:3393, 1989.
Kadesch and Berg, Mol. Cell. Biol., 6:2593, 1986.
Kaeppler et al., Plant Cell Reports, 9:415-418, 1990.
Kaneda et al., Science, 243:375-378, 1989.
Karin at al., Mol. Cell. Biol., 7:606, 1987.
Katinka et al., Cell, 20:393, 1980.
Katinka at al., Nature, 290:720, 1981.
Kato at al, J. Biol. Client., 266:3361-3364, 1991.
Kattman et al., Developmental Cell, 11:723-732, 2006.
Kawamoto at al., Mol. Cell. Biol., 8:267, 1988.
Kelleher and Vos, Biotechniques, 17(6):1110-7, 1994.
Kiledjian et al., Mol. Cell. Biol., 8:145, 1988.
Klamut et al., Mol. Cell, Biol., 10:193, 1990.
Klein et al., Nature, 327:70-73, 1987.
Koc et al., J. Cliff. Oncol., 18:307-316, 2000.
Koch et al., Mol. Cell. Biol., 9:303, 1989.
Kraus et al. FEBS Lett., 428(3):165-170, 1998.
Kriegler and Botchan, In: Eukaryotic Viral Vectors, Y. Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kuhl et al., Cell, 50:1057, 1987.
Kunz et al., Nucl. Acids Res., 17:1121, 1989.
Lareyre et al., J. Biol. Chem., 274(12):8282-8290, 1999.
Larsen et al., Proc. Nat'l Acad. Sci. USA., 83:8283, 1986.
Laspia et al., Cell, 59:283, 1989.
Lastraioli et al., Cancer Res., 64: 606-611, 2004
Latimer et al., Mol. Cell. Biol., 10:760, 1990.
Laughlin et al., J. Virol., 60(2):515-524, 1986.
Lebkowski et al., Mol. Cell. Biol., 8(10):3988-3996, 1988.
Lee et al., DNA Cell Biol., 16(11):1267-1275, 1997.
Lee et al., Nature, 294:228, 1981.
Lee et al., Nucleic Acids Res., 12:4191-206, 1984.
Levenson et al., Hum. Gene Ther., 9(8):1233-1236, 1998.
Levinson et al., Nature, 295:79, 1982.
Lin et al., Mol. Cell. Biol., 10:850, 1990.
Luria et al., EMBO J., 6:3307, 1987.
Lusky and Botchan, Proc. Nat'l Acad. Sci. USA., 83:3609, 1986.
Lusky et al., Mol. Cell. Biol., 3:1108, 1983.
Macejak and Samow, Nature, 353:90-94, 1991.
Majors and Varmus, Proc. Nat'l Acad. Sci. USA., 80:5866, 1983.
Maniatis, et al., Molecular Cloning, A Laboratory Manual, Cold Spring Harbor Press, Cold Spring Harbor, N.Y., 1988.
Mann et al., Cell, 33:153-159, 1983.
Masi et al., Br J. Cancer., 93:781-792, 2005
McLaughlin et al., I Virol., 62(6):1963-1973, 1988.
McNeall et al., Gene, 76:81, 1989.
Messina et al., Circ. Res., 95(9):911-921, 2004.
Miksicek et al., Cell, 46:203, 1986.
Miller et al., Am. J. Clin. Oncol., 15(3):216-221, 1992.
Miyashita et al., Mol Biol Cell., 15:3950-3963, 2004.
Mordacq and Linzer, Genes and Dev., 3:760, 1989.
Moreau et al., Nucl. Acids Res., 9:6047, 1981.
Muesing et al., Cell, 48:691, 1987.
Nabel et al., Science, 244(4910):1342-1344, 1989.
Naldini et al., Science, 272(5259):263-267, 1996.
Ng et al., Nuc. Acids Res., 17:601, 1989.
Nicolas and Rubinstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Nomoto et al., Gene, 236(2):259-271, 1999.
Omirulleh et al, Plant Mol. Biol. 21(3):415-428, 1993.
Ondek et al., EMBO J., 6:1017, 1987.
Ornitz et al., Mol. Cell. Biol., 7:3466, 1987.
Palmiter et al., Nature, 300:611, 1982.
Paskind et al., Virology, 67:242-248, 1975.
PCT Appln. WO 05/054448
PCT Appln. WO 06/017320
PCT Appln. WO 06/078034
PCT Appln. WO 06/080434
PCT Appln. WO 94/09699
PCT Appln. WO 94/09699
PCT Appln. WO 95/06128
Pech et al., Mol. Cell. Biol., 9:396, 1989.
Pelletier and Sonenberg, Nature, 334(6180):320-325, 1988.
Perales et al., Proc. Natl. Acad. Sci. USA, 91:4086-4090, 1994.
Perez-Stable and Constantini, Mol. Cell. Biol., 10:1116, 1990.
Picard and Schaffner, Nature, 307:83, 1984.
Pillozzi et al., Leukemia, 16:1791-1798, 2002.
Pinkert et al., Genes and Dev., 1:268, 1987.
Ponta et al., Proc. Nat'l Acad. Sci. USA., 82:1020, 1985.
Porton et al., Mol. Cell. Biol., 10:1076, 1990.
Potrykus et al., Mol. Gen. Genet., 199(2):169-177, 1985.
Potter at al., Proc. Natl. Acad. Sci. USA, 81:7161-7165, 1984.
Puceat et al., Mol Biol Cell., 14: 2781-2792, 2003
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Resendez Jr. et al., Mol. Cell. Biol., 8:4579, 1988.
Ridgeway, In: Vectors: A Survey of Molecular Cloning Vectors and Their Uses, Rodriguez et al. (Eds.), Stoneham: Butterworth, 467-492, 1988.
Ripe et al., Mol. Cell. Biol., 9:2224, 1989.
Rippe, et al., Mol. Cell. Biol., 10:689-695, 1990.
Rittling et al., Nucl. Acids Res., 17:1619, 1989.
Romanov et al. Stem Cells, 21:105-110, 2003.
Rosen et al., Cell, 41:813, 1988.
Roux et al., Proc. Natl, Acad. Sci. USA, 86:9079-9083, 1989.
Sakai et al., Genes and Dev., 2:1144, 1988.
Sambrook et al., In: Molecular cloning: a laboratory manual, 2^{nd} Ed., Cold Spring Harbor Laboratory Press, Cold Spring Harbor, N.Y., 1989.
Satake et al., J. Virology, 62:970, 1988.
Schachinger at al., N Engl. J. Med., 355:1210-1221, 2006.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985. Smith at al., Circulation, 115(7):896-908, 2007.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Teng et al., Cardiovasc. Res. 61: 268-277, 2004.
Thiesen et al., J. Virology, 62:614, 1988.
Tratsehin et al., Mol. Cell. Biol., 4:2072-2081, 1984.
Treisman, Cell, 42:889, 1985.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsumaki et al., J. Biol. Chem., 273(36):22861-22864, 1998.
Tur-Kaspa et al., Mol. Cell. Biol., 6:716-718, 1986.
Tyndall et al., Nuc. Acids. Res., 9:6231, 1981.
Vannice and Levinson, J. Virology, 62:1305, 1988.
Vasseur et al., Proc. Nat'l Acad. Sci. USA., 77:1068, 1980.
Wagner et al., Proc. Natl. Acad. Sci. USA 87(9):3410-3414, 1990.
Wang and Calame, Cell, 47:241, 1986.
Wang et al., Cancer Res., 62:4843-4848, 2002
Wang et al., J Biol. Chem., 279: 13289-13292, 2004.
Weber et al., Cell, 36:983, 1984.
Weinberger et al. Mol. Cell. Biol., 8:988, 1984.
Wilson et al., Science, 244:1344-1346, 1989.
Winoto and Baltimore, Cell, 59:649, 1989.
Wollert et al., Lancet, 364:141-148, 2004.
Wong et al., Gene, 10:87-94, 1980.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Wu et al., Biochem. Biophys. Res. Commun., 233(1):221-226, 1997.
Yang and Russell, Proc. Natl. Acad. Sci. USA, 87:4144-4148, 1990.
Yutzey et al. Mol. Cell. Biol., 9:1397, 1989.
Zhao-Emonet et al., Biochim. Biophys. Acta, 1442(2-3):109-119, 1998.
Zufferey et al., Nat. Biotechnol., 15(9):871-875, 1997.

1. A mesodermal progenitor cell capable of differentiation into a mesodermal lineage comprising an expression cassette comprising a wild-type (wt) ERG coding region under the control of a heterologous promoter integrated into the genome of the cell.

2. The mesodermal progenitor call of claim 1, where in the mesodermal lineage is a cardiac or vascular tissue lineage.

3. The mesodermal progenitor cell of claim 1, wherein the cell expresses increased levels of wt-ERG polypeptide relative to normal cardiomyocytes.

4. The mesodermal progenitor cell of claim 1, further defined as a population of cardiac progenitor cells comprising beating embryoid bodies.

5. The mesodermal progenitor cell of claim 1, wherein the cell displays cardiomyocyte markers.

6. The mesodermal progenitor cell of claim 1, wherein the cell displays endothelial cell markers.

7. The mesodermal progenitor cell of claim 6, wherein the cell is further defined as CD31 positive cell.

8. The mesodermal progenitor cell of claim 1, further defined as a population of mesodermal progenitor cells comprising cells with endothelial markers and cells with cardiomyocyte markers.

9. The mesodermal progenitor cell of claim 1, wherein the cell is a human cell.

10. The mesodermal progenitor cell of claim 1, wherein the heterologous promoter is a viral promoter or an inducible promoter.

11. The mesodermal progenitor cell of claim 10, wherein the heterologous promoter is a CMV promoter.

12. The mesodermal progenitor cell of claim 1, wherein the wt-ERG is a human wt-ERG (wt-HERG).

13. The mesodermal progenitor cell of claim 1, wherein the expression cassette further comprises a selectable marker or a reporter gene.

14. The mesodermal progenitor cell of claim 13, wherein the reporter gene is GFP.

15. The mesodermal progenitor cell of claim 1, wherein the cell is a cardiac progenitor cell and has a hyperpolarized resting membrane potential.

16. The mesodermal progenitor cell of claim 15, wherein the cell has a resting membrane potential of less than about −70 mV.

17. The mesodermal progenitor cell of claim 1, further defined as a population of cardiac progenitor cells having shortened action potentials relative to cells that do not comprise the wt-ERG expression cassette.

18. The mesodermal progenitor cell of claim 1, further defined as a population of cardiac progenitor cells comprising homogeneous action potentials.

19. The mesodermal progenitor cell of claim 1, wherein the cells are adherent in culture.

20. A method for making mesodermal progenitor cells comprising:
(a) obtaining a pluripotent cell population from a mammal;
(b) transforming at least one progenitor cell with an expression cassette comprising a wt-ERG coding region under the control of a heterologous promoter; and
(c) allowing the cells grow under permissive conditions and thereby differentiate into mesodermal progenitor cells.

21. The method of claim 20, further comprising the step of (c) selecting transformed cells that comprise the expression cassette after transforming at least one progenitor cell with the expression cassette.

22. The method of claim 20, wherein the progenitor cell population is an embryonic stem cell population.

23. The method of claim 20, wherein the progenitor cell population is a cord blood stem cell population or a mesenchymal cord stem cell population.

24. The method of claim 20, wherein the progenitor cell population is a bone marrow cell population.

25. The method of claim 20, wherein the progenitor cell population is a cardiac stem cell population or an adult cardiac progenitor cell.

26. The method of claim 20, wherein the transforming comprises transfection of the cells.

27. The method of claim 20, wherein the transforming comprises transduction of the cells viral vector.

28. The method of claim 20, wherein the expression cassette is a plasmid or viral vector.

29. The method of claim 28, wherein the viral vector is an adenovirus or retroviral vector.

30. The method of claim 29, wherein the retroviral vector is a lentiviral vector.

31. The method of claim 20, wherein the heterologous promoter is a viral promoter or an inducible promoter.

32. The method of claim 31, wherein the heterologous promoter is a CMV promoter.

33. The method of claim 20, wherein the wt-HERG coding region is a human HERG coding region.

34. The method of claim 20, wherein the selection of cells is further defined as selecting cells comprising the wt-ERG expression cassette integrated into their genome.

35. The method of claim 20, wherein the expression cassette comprises a drug resistance marker and the selection of cells is a drug selection.

36. The method of claim 35, wherein the drug is G418.

37. The method of claim 20, wherein the expression cassette comprises a reporter gene and the selection of cells is a drug selection is by detection of the reporter gene.

38. The method of claim 37, wherein the reporter gene is a fluorescence protein.

39. The method of claim 38, wherein the selection is by FACS.

40. The method of claim 20, wherein allowing the cells grow under permissive conditions and thereby differentiate into mesodermal progenitor cells comprises growing the cells in a medium lacking leukemia inhibitory factor.

41. The method of claim 20, wherein allowing the cells grow under permissive conditions and thereby differentiate into mesodermal progenitor cells comprises growing the cells in a gel matrix.

42. The method of claim 20, wherein allowing the cells grow under permissive conditions and thereby differentiate into mesodermal progenitor cells comprises growing the cells in co-culture with stromal cells.

43. A cell produced by the method of claim 20.

44. The method of claim 20, further defined as a method for producing a mesodermal tissue and further comprising:
(d) growing the mesodermal progenitor cells under conditions that are permissive for mesodermal tissue formation.

45. The method of claim 44, wherein the mesodermal tissue is a cardiac tissue.

46. The method of claim 44, wherein the mesodermal progenitor cells are grown on a scaffold or matrix.

47. A method for treating a patient with myocardial damage comprising:
obtaining a population of mesodermal progenitor cells according to claim 1, or a population of cells made by the method of claim 20;
(ii) administering an effective amount of the cells into the patient, thereby allowing the cells to integrate into the patients myocardial tissue.

48-60. (canceled)


SEQUENCE LISTING (1 sequence)

SEQ ID NO: 1 - protein, 1159 aa, Homo sapiens [sequence omitted]